(19) United States
(12) Patent Application Publication
(10) Pub. No.: US 2022/0387759 A1
(43) Pub. Date: Dec. 8, 2022

(54) INTRODUCER LOCK APPARATUS

(71) Applicant: Boston Scientific Scimed, Inc., Maple Grove, MN (US)

(72) Inventors: Michael Turovskiy, Fremont, CA (US); Jayson Delos Santos, Fremont, CA (US); Michael P. Wallace, Fremont, CA (US)

(73) Assignee: Boston Scientific Scimed, Inc., Maple Grove, MN (US)

(21) Appl. No.: 17/830,878

(22) Filed: Jun. 2, 2022

Related U.S. Application Data
(63) Continuation of application No. 63/196,209, filed on Jun. 2, 2021.

(51) Int. Cl. A61M 25/02

(52) U.S. Cl. CPC A61M 25/02; A61M 2025/0006

(57) ABSTRACT

Apparatuses and methods for retaining a catheter are disclosed. In particular, described herein are apparatuses (e.g., introducer lock apparatuses, devices and systems including them) and methods for securing a catheter relative to an introducer sheath. In particular these apparatuses and method may secure an outer catheter of a mechanical thrombectomy apparatus to prevent the outer catheter from moving proximally when an inner member within the outer catheter is pulled proximally, applying a compression force that would otherwise drive the outer catheter proximally.

### CROSS REFERENCE TO RELATED APPLICATIONS

This application claims the benefit of and priority to U.S. Provisional Patent Application Ser. No. 63/196,209 filed on Jun. 2, 2021, the disclosure of which is incorporated herein by reference.

### INCORPORATION BY REFERENCE

All publications and patent applications mentioned in this specification are herein incorporated by reference in their entirety to the same extent as if each individual publication or patent application was specifically and individually indicated to be incorporated by reference.

### BACKGROUND

Minimally invasive medical procedures may be preferred by clinicians and patients because of a faster healing time and decreased pain. For example, minimally invasive procedures that use a catheter assembly may need only small incisions to enter the body, blood vessel, or other body lumen. Small incisions may be less painful and heal more rapidly than larger incisions, especially large incisions that open up major portions of the patient's body.

A catheter may be guided to a treatment area through one or more small incisions and medical treatment provided. Following treatment, the catheter may be withdrawn, and any incisions closed. As part of the medical procedure, a distal end of the catheter may be coupled to tools that may be controlled by one or more wires, shafts, or tendons within the catheter. In some cases, manipulation of the wires, shafts, or tendons may require moving them relative to an outer catheter.

In particular, devices for mechanically removing material, including thrombus material, from within a lumen of the vessel may include a catheter over which an inverting tube may be used to remove material from within the body lumen. For example, these devices may be used as mechanical thrombectomy catheters for removing a clot from a blood vessel (e.g., thrombectomy devices), similar to those disclosed and described in each of U.S. Pat. No. 10,271,864, as well as in each of U.S. Patent Application Publication Nos. 2019/0117214, 2018/0042626 and 2018/0042624, and in U.S. patent application Ser. No. 16/566,393. These apparatuses do an excellent job at removing material from within a blood vessel but may require multiple hands to operate.

Thus, there is a need for devices and systems including these devices, that can be used to help control catheters, an in particular catheters in which a flexible tube is inverted and rolled over or at the distal end of the catheter, to enhance the ease of use and precision with which these apparatuses may be operated. Described herein are apparatuses (devices, systems and kit) and methods of using them that may address the needs and problems discussed above.

### SUMMARY OF THE DISCLOSURE

Described herein are apparatuses and methods for releasably securing a catheter (including in particular a mechanical thrombectomy catheter) relative to an introducer sheath to prevent the catheter from moving proximally out the patient even when a proximally-directed (e.g., compressive) force is applied on the catheter. These methods and apparatuses may be particularly helpful for mechanical atherectomy apparatuses.

In general, the apparatuses and methods described herein may include a first body that is configured to attached at the distal end to an introducer sheath securely and removably. The first body may include a catheter channel that runs in the same axis as the sheath, so that a portion of an outer catheter within introducer may reside in the catheter channel. A second body is coupled to the first body at the proximal end region by a hinge that allows the second body, which may be an arm, lever, etc. to pivot relative to the first body and the introducer sheath. The second body may pivot from an axis that is parallel with the first body (and with the introducer sheath, in which the angle between the first body and the second body is 180 degrees at the hinge region) to form an angle (e.g., between 180 degrees and 10 degrees, between 180 degrees and 40 degrees, between 180 degrees and 85 degrees, between 180 degrees and 130 degrees, etc. at the hinge region, such as between 180 degrees and 135 degrees at the hinge region). The second body may include a cut-out region forming a clamping channel (also referred to herein as a locking channel) into which the outer catheter may be held. The clamping channel is configured so that when the outer catheter extending from/into the sheath is held in the catheter channel and in the clamping channel, any movement of the outer catheter proximally (e.g., out of the sheath), as from the application of a compressive force on the outer catheter when pulling an inverting tube over the distal end the outer catheter, may cause the angle between the first body and the second body to decrease, clamping the clamping channel onto the outer catheter, and preventing it from moving further proximally.

The introducer lock apparatus may be applied onto the introducer sheath when the outer catheter is already inserted into the introducer sheath, and may engage (e.g., lock onto) an attachment site on the introducer.

In general, the introducer lock apparatuses and methods of using them described herein may allow the catheter apparatus, such as a mechanical thrombectomy apparatus, to be operated using just two hands; alleviating the need for the operator or an assistant to hold the outer catheter fixed relative to the introducer. Instead, the user may operate a mechanical thrombectomy apparatus with just two hands, for example, holding the introducer sheath with one hand and operating the inner member (e.g., a puller or inner catheter) within the outer catheter with the other hand.

For example, described herein are introducer lock apparatuses. Any of these apparatuses may include: a first body comprising a catheter channel extending proximally to distally in a first axis, wherein the catheter channel is configured to seat a catheter therein; a sheath connector at a distal end of the first body, the sheath connector at least partially surrounding the catheter channel and is configured to releasably engage with an introducer sheath (e.g., with a handle portion of an introducer); a second body hingeably coupled to the first body; and a clamping channel extending through a lateral side of the second body, the clamping channel forming an angle relative to the second body, so that the clamping channel has an apparent diameter through the clamping channel in the first axis that is at a maximum when the second body is bent at a first angle relative to the first body, further wherein the apparent diameter through the clamping channel in the first axis decreases as the angle relative to the second body increases.

In any of the apparatuses described herein, the second body may include one or more teeth extending into the clamping channel from a wall of the clamping channel. In any of these apparatuses, the second body may include a tooth extending out of the clamping channel and projecting from an outer surface of the second body at a locking angle. The teeth may be pointed or rounded. The teeth may extend into the channel, in some examples, the teeth extend only partially into the channel. The locking angle may be, for example, between 15 degrees and 100 degrees (e.g., between 20 degrees and 95 degrees, between 30 degrees and 65 degrees, between 40 degrees and 50 degrees, etc.) relative to the long axis of the second body (e.g., the arm axis).

In any of these apparatuses, a central axis extending along the clamping channel may be parallel with the first axis when the apparent diameter is at the maximum.

The catheter channel may be open along an outward-facing side of the first body. For example, the side of the channel opposite from the first body may be open. Thus, in some examples, the channel may extend between 30-60% around the outer catheter (e.g., about 55%, about 50%, about 45%, about 40%, etc. around the outer catheter) when the outer catheter is held within the catheter channel. In some examples the channel may be fully enclosed in the channel (e.g., 100% enclosed). The inner diameter of the channel may be larger than the outer diameter of the outer catheter, so that the outer catheter may otherwise (but for the clamping channel) freely slide within the channel. In some examples in which the channel is closed over the outer catheter, the first body may include a second body that closes over the channel, forming the fully enclosed channel. In some examples in which the channel is closed over the outer catheter, the catheter may be slide into the channel, e.g., by threading over the channel.

Although the examples described herein refer to “outer catheter” these apparatuses may be used with any channel. For convenience, the examples herein refer specifically to an “outer catheter” of a mechanical thrombectomy apparatus.

The clamping channel is configured to hold the outer catheter with a clamping force that depends on the angle of the second body relative to the first body. Thus, when the user (e.g., physician, clinician, etc.) wants to advance the outer catheter distally (or intentionally withdraw it proximally) the user may hold the second body fixed relative to the first body (e.g., at a predetermined angle of, e.g., 135 degrees), allowing the outer catheter to move distally or proximally without changing the relative angle of the second body, and preventing the second body from clamping the outer catheter in position by narrowing the apparent diameter through the clamping channel.

In any of these examples, the clamping channel forms a C-shaped channel having a side opening. The side opening may not align with the opening of the catheter channel (helping prevent the outer catheter from falling out of the clamping channel and/or the catheter channel, particularly when changing the angle of the second body to clamp the outer catheter).

The connector (the sheath connector) portion of the first body may be a separate portion that is coupled to the first body or it may be integrally formed with the sheath body. In some cases, the sheath connector only partially surrounding the catheter channel. For example, the sheath connector may be a partial Leur fitting (e.g., a c-shaped, rather than fully circular Leur fitting). In some cases, the sheath connector may be a Leur fitting. Any of these apparatuses may include a stop on the first body, that is configured to limit the angle between the first body and the second body. For example, the stop may prevent the second body from bending from forming an angle relative to the first body of less than 130 degrees (e.g., less than 132 degrees, less than 135 degrees, less than 140 degrees, etc.).

In some examples, the first angle relative to the first body is between 120-130 degrees (e.g., about 135 degrees) at the hinge (hinge region).

As mentioned, the second body may also be configured as an arm or lever. The second body is generally rigid.

Any of these apparatuses may include a bias configured to hold the second body at the first angle relative to the first body. For example, the bias may be a spring bias. The bias may be configured to hold the second body at an angle of between 120-130 degrees relative to the first body.

Also described herein are methods of operating any of these apparatuses. For example, also described herein are methods of securing a catheter (e.g., outer catheter), including methods of operating a mechanical atherectomy system. For example, a method may include; advancing a distal end of an outer catheter of the mechanical atherectomy system through a sheath and into a body lumen; securing the outer catheter within an introducer lock apparatus coupled to the proximal end of the sheath so that a first region of the outer catheter is seated in a catheter channel of a first body extending in a first axis, and a second region of the outer catheter proximal to the first region is held within a clamping channel of the introducer lock apparatus, wherein the clamping channel is formed through a lateral side of a second body that is hingeably coupled to the first body; pulling an inner shaft of the mechanical atherectomy system proximally to invert a tubular member over a distal end of the outer catheter so that it rolls over the distal end of the outer catheter and into the outer catheter; and locking the outer shaft to the sheath by changing the angle of the second body when the inner shaft is driven proximally by pulling the inner shaft, so that an apparent diameter through the clamping channel in the first axis decreases.

In any of these methods, pulling the inner shaft proximally may apply a proximal force on the outer catheter that changes the angle of the second body to lock the outer shaft to the sheath.

Locking the outer shaft to the sheath may comprise gripping an outer surface of the outer catheter with one or more teeth extending from a wall of the clamping channel and into the clamping channel. In some examples, locking the outer shaft to the sheath comprises gripping an outer surface of the outer catheter with one or more teeth extending projecting from an outer surface of the second body at a locking angle.

As mentioned, the method may be performed by a user holding the sheath with a first hand and a proximal end of the inner member with a second hand. The user may not hold the outer catheter.

Locking the outer shaft to the sheath may comprise gripping one or more stops on an outer surface of the outer catheter. Any of these methods may include advancing the outer catheter distally through the sheath. As mentioned, in some examples, advancing the outer catheter distally may include holding the angle between the first body and the second body fixed (e.g., at the first angle, having the maximum apparent diameter through the clamping channel.

In general, advancing the outer catheter distally through the sheath may include advancing the outer catheter distally through the sheath while otherwise held by the sheath connector. In any of the apparatuses described herein, the sheath connector may be configured to allow the catheter to advance distally (e.g., further into the patient's body) while preventing the outer catheter from retracting proximally, even when column force is applied from the distal end of the outer catheter as the inverting tube is pulled over the distal end. The sheath connector, and in particular the second body (e.g., arm) of the sheath connector may be configured to prevent the outer catheter from pulling proximally yet still allow it to be advanced distally. In some examples the sheath connector may include a stop (e.g., a “neutral stop”) that prevents the second body of the sheath connector from bending further relative to the first body (e.g. from reducing the angle between the second body and the first body) when the outer catheter is pushed distally. Alternatively or additionally, in some examples the user may use her or his finger to hold the second body in a predetermined neutral position to allow the outer catheter to be slide distally or in some cases, proximally). The sheath connector may include an indicator or grip to guide the user to hold the second body in this neutral position (e.g., with the angle between the first body and the second body at a predetermined angle, such as 135 degrees).

In general, any of these methods may include coupling the introducer lock apparatus to the proximal end of the sheath. In some examples the introducer lock apparatus may be coupled to the proximal end of the sheath while the outer catheter is extending out of the sheath (e.g., after the outer catheter has been positioned within the sheath and therefore within the body).

In any of these apparatuses, the catheter may be restricted or limited in one direction when an inner shaft of the catheter is moved in a similar direction.

Thus, in general the apparatuses and methods may include an introducer lock apparatus that may be removably attached to an introducer sheath used to control or guide the catheter. The introducer lock apparatus may preferentially clamp the catheter as the catheter moves in a first direction. In some cases, the introducer lock apparatus may clamp and/or restrict catheter motion in a first direction but not a second direction.

The introducer lock apparatus may include a first body and a second body. The first body may be removably attached to the introducer sheath and the second body may by hingeably coupled to the first body. The first body may be colinear and/or coplanar with respect to the catheter when attached to the introducer sheath. In some examples, the first body may include an indicator (e.g., an arrow, etc.) on the first body to guide the user in attaching to the introducer sheath. In some variations, a bias, such as a spring, may bias a position of the second body with respect to the first body (e.g., in a preferred angle).

The second body may include a clamping channel that receives the catheter. As the catheter moves in a first direction (proximally, for example), the clamping channel may engage (clamp) the catheter. As the catheter moves in a second direction (distally, for example), the clamping channel may no longer clamp the catheter. In some cases, motion in the second direction may reduce a clamping force applied to the catheter.

As used herein, the proximal direction may be generally understood as towards the user operating the apparatus, while the distal direction may generally be understood as away from the user operating the apparatus.

The clamping channel may be implemented as a three-sided slot within the second body. In some variations, the three-sided slot may be disposed at an angle (e.g., a forty-five degree angle) with respect to an outer surface of the second body. In some variations, the clamping channel may include one or more teeth to engage and/or clamp an outer catheter.

The first body may attach to the introducer sheath with locking lugs that couple and/or engage with a female channel in the introducer sheath. In some variations, the first body may be rotated a quarter-turn to attach the first body to the introducer sheath.

In some variations, the outer catheter may include stops that engage with the clamping channel. The stops may be molded or otherwise attached to the outer catheter. The stops may be slightly larger than the outer catheter and enable the clamping channel to have an increased grip with respect to the catheter.

One innovative aspect of the subject matter described herein can be implemented as a method of operating a catheter system. The method may include advancing a distal end of a catheter through a body lumen, where the catheter includes an elongate body surrounding at least one inner shaft, receiving the catheter (e.g., outer catheter) into a clamping channel of an introducer lock apparatus, wherein a first body of the introducer lock apparatus is coupled to an introducer sheath and a second body of the introducer lock apparatus is hingeably coupled to the first body and includes the clamping channel. Furthermore, the method may include moving the inner shaft in a proximal direction with respect to the introducer sheath and engaging the outer catheter with the clamping channel in response to proximal motion of the inner shaft. The proximal motion of the inner shaft may increase engagement of the outer catheter with the clamping channel.

In some examples, the clamping channel may include a three-sided slot within the second body. Furthermore, two parallel sides of the three-sided slot are disposed at approximately a forty-five degree angle with respect to an outer surface of the second body.

In some examples, the clamping channel may include a plurality of teeth to engage the outer catheter. Furthermore, at least one of the teeth of the clamping channel may be disposed at approximately ninety degrees with respect to a surface of the clamping channel.

In some examples, the engaging may include the clamping channel engaging with one or more stops molded onto the outer catheter. In some other examples, the method may include moving the second body of the introducer lock apparatus in the proximal direction in response to moving the inner shaft in the proximal direction.

In some examples, the method may include advancing the outer catheter distally through the introducer sheath, where advancing the outer catheter disengages the outer catheter from the clamping channel. In some other examples, the first body of the introducer lock apparatus may be removably attached to the introducer sheath. In some cases, the first body of the introducer lock apparatus may be coupled to the second body via a hinge joint allowing motion in only one plane. Furthermore, the catheter may include a plurality of inner shafts.

Another innovative aspect of the subject matter described herein can be implemented as a catheter retaining apparatus. The apparatus may include a catheter having a distal end configured to be advanced through a body lumen, where the catheter (e.g., outer catheter) surrounds at least one inner shaft and an introducer lock apparatus comprising a first body hingeably coupled to a second body. The first body may be configured to be coupled to an introducer sheath and the second body may be configured to engage the outer catheter within a clamping channel, where proximal motion of the inner shaft relative to the introducer sheath increases engagement of the outer catheter with the clamping channel.

In some examples, the clamping channel may include a three-sided slot within the second body. Furthermore, two parallel sides of the three-sided slot may be disposed at approximately a forty-five degree angle with respect to an outer surface of the second body.

In some examples, the clamping channel may include a plurality of teeth configured to engage the outer catheter. Furthermore, at least one of the teeth of the clamping channel may be disposed at approximately ninety degrees with respect to a surface of the clamping channel.

In some examples, the outer catheter may include one or more molded stops configured to engage with the clamping channel. In some other examples, the second body of introducer lock apparatus may be configured to move in a proximal direction in response to proximal motion of the inner shaft.

The clamping channel may be configured to disengage with the outer catheter as the outer catheter is advanced distally with respect to the introducer sheath. Furthermore, the first body of the introducer lock apparatus may be configured to removably attach to the introducer sheath.

In some examples, the first body of the introducer lock apparatus may be coupled to the second body via a hinge joint configured to allow motion in only one plane. Furthermore, the catheter may include a plurality of inner shafts. In some examples, the first body may include an arrow configured to guide the user to couple the first body to the introducer sheath.

### BRIEF DESCRIPTION OF THE DRAWINGS

Novel features of embodiments described herein are set forth with particularity in the appended claims. A better understanding of the features and advantages of the embodiments may be obtained by reference to the following detailed description that sets forth illustrative embodiments and the accompanying drawings.

FIG. 1 illustrates an example of a system including a mechanical thrombectomy apparatus (e.g., outer catheter, inverting tube, and puller, along with a guidewire), as well as an introducer sheath and an introducer lock apparatus as described herein. The system is shown within a blood vessel proximate to a clot.

FIGS. 2A-2C illustrate an example of a mechanical thrombectomy apparatus as described herein, including an inverting tube that may be used to remove material from a vessel. In FIG. 2A, the assembled apparatus is shown in a side view, showing an inversion support catheter and a flexible outer tube. FIG. 2B shows the inverting tube apparatus of FIG. 2A in a vessel, proximal to a clot. FIG. 2C illustrates the removal of a clot from the vessel using the apparatus of FIG. 2A, by pulling the flexible tube on the outside of the inversion support catheter proximally so that it rolls over the distal end of the inversion support catheter and into the inversion support catheter, drawing the clot with it; the apparatus may be advanced distally.

FIG. 3A shows a first view of a catheter controlling system, in accordance with some examples.

FIG. 3B shows a second view of the catheter controlling system, in accordance with some examples.

FIG. 4A shows a cross sectional view of a catheter that may include an inner shaft.

FIG. 4B shows a side view of a catheter, in accordance with some examples.

FIG. 5A shows a first view of a catheter controlling system, in accordance with some examples.

FIG. 5B shows a second view of the catheter controlling system.

FIG. 6A shows a first view of an introducer lock apparatus, in accordance with some examples.

FIG. 6B shows a second view of the introducer lock apparatus, in accordance with some examples.

FIG. 6C shows a third view of the introducer lock apparatus, in accordance with some examples.

FIG. 7A shows a first view of a first body of the introducer lock apparatus of FIGS. 6A-6C.

FIG. 7B shows a second view of the first body of the introducer lock apparatus of FIGS. 6A-6C.

FIG. 7C shows a third view of the first body of the introducer lock apparatus of FIGS. 6A-6C.

FIG. 8A shows a first view of a second body of the catheter retainer lock of FIG. 6A-6C.

FIG. 8B shows a second view of the second body of the catheter retainer lock of FIGS. 6A-6C.

FIG. 8C shows a third view of the second body of the catheter retainer lock of FIGS. 6A-6C.

FIG. 9 is a flowchart depicting an example of one method for controlling a catheter.

### DETAILED DESCRIPTION

Minimally invasive medical procedures often use catheter-based systems to provide therapy that would otherwise require more traumatic methods. The smaller incisions associated with catheter therapies that are used to enter the body may promote faster healing and lessen the recovery time for the patient. During minimally invasive procedures, a distal end of the catheter may be used to manipulate various tools or internal organs while the clinician operates at a distance. In some cases, the clinician may use robotics to manipulate or control the catheter.

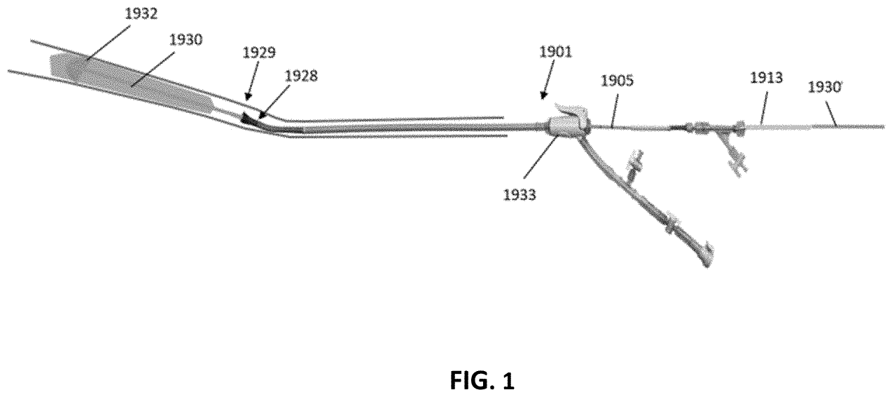

FIG. 1 illustrates one example of a system including both a mechanical thrombectomy apparatus 1901 (also referred to herein as an inverting thrombectomy apparatus) an introducer 1933. In FIG. 1, the mechanical thrombectomy apparatus 1901 is shown deployed within a vessel proximate to a clot 1930, 1930′. In this example, an optional expandable scraper 1932 is shown deployed through the inverting thrombectomy apparatus and into the clot, and expanded within the clot, as described above. The inverting thrombectomy apparatus generally includes any of the features discussed above, including an inversion support catheter, a flexible (e.g., knitted) tube 1928 inverted over the distal end 1929 of the inversion support catheter, and a puller 1913. In this example, an optional expandable scraper 1932 is deployed through the inverting thrombectomy apparatus (e.g., through the flexible tube and inversion support catheter) and includes a distal expanding (scraping) region (“distal expanding region”) 1932. The inverting thrombectomy apparatus and expandable scraper may be deployed through a sheath hub 1933.

The outer catheter 1905 may include multiple elements (control wires, shafts, etc.) to manipulate and/or articulate objects at the distal end. In particular, the methods and apparatuses for controlling a catheter described herein may be useful for preventing undesirable movement in an outer catheter of the mechanical thrombectomy apparatus. For example, a medical procedure may require the clinician to move portions of the catheter separately. A mechanical thrombectomy apparatus may have an inner catheter 1905 having a lumen holding an inner shaft 1913 (e.g., puller). Manipulation of a tool may require that the inner shaft move with respect to the outer catheter. In some cases, providing the relative motion may require holding the outer catheter while at the same time moving the inner shaft. However, the clinician may also need to guide the catheter at the same time, making control of the catheter-based system difficult.

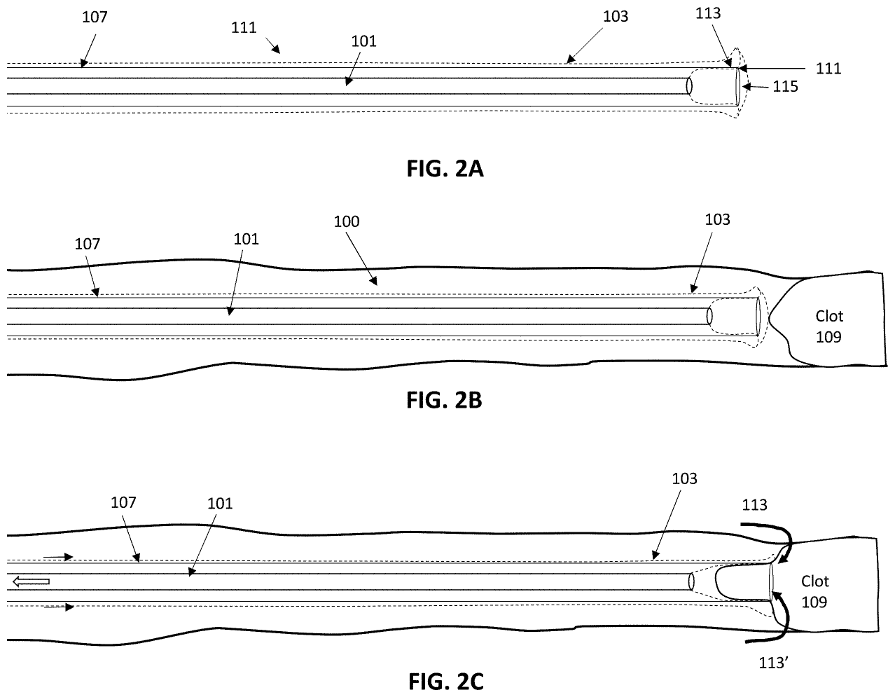

In general, an inverting tube apparatus (also referred to herein as “mechanical thrombectomy apparatus” or “inverting thrombectomy apparatus”) may be configured to remove material, such as clot, using a length of inverting tube, as shown in FIGS. 2A-2C. The apparatuses and methods of using them described herein may be used within the vasculature, including the neurovasculature and the peripheral vasculature.

For example, FIG. 2A illustrates an example of an inverting thrombectomy apparatus 100, such as described in U.S. patent application Ser. No. 15/496,570, and in U.S. Pat. No. 9,463,035. The apparatus includes an inversion support catheter 107 and a flexible tube 103 that extends over the outer surface of the inversion catheter. The flexible tube may be referred to as a tractor tube (or flexible tractor tube) and may be attached at one end region to a puller 101, which may be pull wire or pull tube (e.g., catheter), e.g., at the distal end region of the puller. In some examples, the flexible tube may be attached proximal to the distal end of the puller (e.g. between 1 mm and 50 mm from the distal end, between 1 mm and 40 mm, between 1 mm and 30 mm, greater than 5 mm, greater than 10 mm, greater than 20 mm, greater than 30 mm, etc. from the distal end of the puller). Pulling the puller proximally inverts the flexible tube over the distal end opening 111 of the inversion support catheter to capture and remove a material (such as a clot) in the vessel lumen, as shown in FIGS. 2B and 2C. In operation, the amount of the material that may be captured corresponds to the length of the flexible tube.

In FIG. 2B the inverting tractor mechanical thrombectomy apparatus 100 is shown deployed near a clot 109. In the deployed configuration the puller 101 (shown here as a puller micro catheter, alternatively the puller may be a wire) is held within an elongate inversion support catheter 107 so that the flexile tractor tube 103 extends from the end of the puller 101 and expands toward the inner radius of the elongate inversion support catheter 107; at the distal end opening 111 of the elongate inversion support catheter the tractor tube inverts over itself and extends proximally in an inverted configuration over the distal end of the elongate inversion support catheter. As shown in FIG. 2C, by pulling the puller proximally, the tractor tube rolls 113, 113′ and everts over the distal end opening of the elongate inversion support catheter, drawing the adjacent clot into the elongate inversion support catheter, as shown.

FIG. 2A the elongate inversion support catheter is an elongate tube having a distal end that has the same size inner diameter as the proximal length of the inversion support catheter. In some examples the distal end of the inversion support catheter may be funnel-shaped (or configured to expand into a funnel shape, see, e.g. FIGS. 2A-2B). In FIGS. 2A-2C, the inversion support catheter 107 is shown positioned between the tractor tube (e.g., flexible tube 103) and the puller 101 so that the flexible tube can be pulled proximally by pulling on the puller and rolling the flexible tube into the elongate inversion support catheter so that it inverts. The portion of the flexible tube that is inverted over the distal end of the elongate inversion support catheter has an outer diameter that is greater than the outer diameter of the elongate inversion support catheter. The flexible tube may be biased so that it has a relaxed expanded configuration with a diameter that is greater than the outer diameter (OD) of the elongate inversion support catheter; in addition, the flexible tube may also be configured (e.g., by heat setting, etc.) so that when the flexible tube is everted and rolled over the distal end opening into the elongate inversion support catheter, the outer diameter of the flexible tube within the elongate inversion support catheter has an outer diameter that is about y times (y fold) the inner diameter of the elongate inversion support catheter (e.g., where y is greater than 0.1×, 0.5×, 0.6×, 0.7×, 0.75×, 0.8×, 0.9×, 1×, etc. the inner diameter, ID, of the elongate inversion support catheter. This combination of an un-inverted diameter of the flexible tube of greater than the diameter of the OD of the elongate inversion support catheter and an inverted diameter of the flexible tube of greater than, e.g., 0.7× the ID of the elongate inversion support catheter is surprisingly helpful for preventing jamming of the apparatus, both when deploying the apparatus and when rolling the flexible tube over the distal end opening of the elongate inversion support catheter to grab a clot. The flexible tube may be expandable and may be coupled to the puller as shown. In some examples the flexible tube and the puller may comprise the same material, but the flexible tube may be more flexible and/or expandable, or may be connected to elongate puller (e.g., a push/pull wire or catheter). As mentioned above, the puller may be optional (e.g., the flexible tube may itself be pulled proximally into the inversion support catheter).

In FIG. 2C the clot may be drawn into the elongate inversion support catheter by pulling the flexible tube proximally and inverting the inverting tube 103 of the thrombectomy apparatus 100 into the distal end of the elongate inversion support catheter 107, as indicated by the arrows 113, 113′ showing pulling of the inner portion of the flexible tube, resulting in rolling the flexible tube over the end opening of the catheter and into the catheter distal end and inverting the expandable distal end region so that it is pulled into the catheter, shown by arrows. The end of the flexible tube outside of the catheter may be loose relative to the outer wall of the catheter.

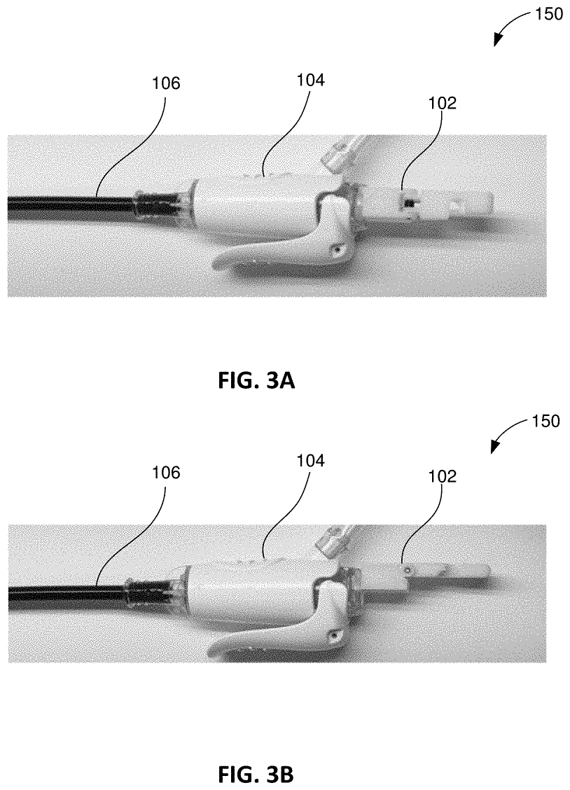

In general, the mechanical thrombectomy apparatuses described in FIGS. 2A-2C may be used with an introducer sheath (as shown in FIG. 1), as well as any of the introducer lock apparatuses as described in greater detail below. For example, FIGS. 3A-3B shows one example of an introducer lock apparatus coupled with an introducer sheath.

Examples of the subject matter described in this disclosure may be used to temporarily grip or restrain a portion of the outer catheter enabling the clinician greater control of the mechanical thrombectomy apparatus. Some examples may include an introducer lock apparatus that may be removably attached to an introducer sheath. The introducer lock apparatus may include a first body hingeably coupled to a second body. A catheter may pass through the introducer sheath and a clamping channel of the second body. As the catheter moves proximally with respect to the introducer sheath, the clamping channel and the second body may increase engagement with an outer portion of the catheter thereby clamping the catheter.

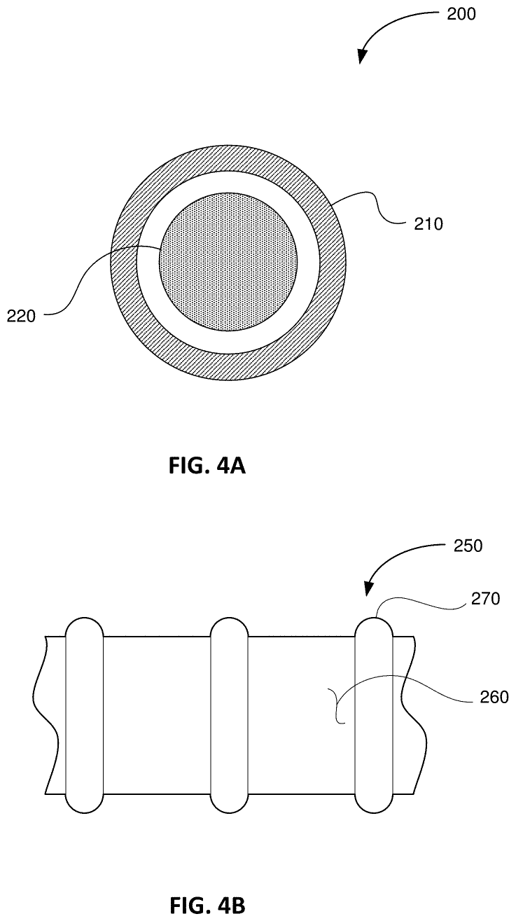

FIG. 3A shows a first view of a catheter controlling system 150, in accordance with some examples. The catheter controlling system 150 may include an introducer lock apparatus 102, an introducer sheath 104, and a sheath 106 (the sheath is the distal portion of the introducer sheath in this example). In some examples, the introducer lock apparatus 102 may be removably attached to the introducer sheath 104. For example, the introducer lock apparatus 102 may include one or more male locking lugs (not shown) that engage with one or more female channels (also not shown) in the introducer sheath 104. In some examples, the male locking lugs and the female channels may enable the introducer lock apparatus 102 to be attached with a quarter-turn (e.g., about ninety degrees) of rotation of the introducer lock apparatus 102 with respect to the introducer sheath 104. For example, the introducer lock apparatus 102 may be rotated one quarter-turn clockwise with respect to the introducer sheath 104 to removably attach the introducer lock apparatus 102 to the introducer sheath 104. The sheath 106 may be coupled to the introducer sheath 104. In some examples, the sheath 106 may receive and guide a catheter (not shown) into a blood vessel or other body lumen of a patent. The catheter may be part of a mechanical thrombectomy apparatus including an outer catheter that encloses at least an inner shaft as described in more detail below with respect to FIGS. 4A and 4B.

The introducer lock apparatus 102 may be initially inserted into the introducer sheath 104, as shown in the first view of a catheter controlling system 150. That is, the introducer lock apparatus 102 may not yet have been rotated a quarter-turn with respect to the introducer sheath 104. Thus, the introducer lock apparatus 102 may not yet be locked into the introducer sheath 104.

FIG. 3B shows a second view of the catheter controlling system 150, in accordance with some examples. As shown, the introducer lock apparatus 102 has been rotated clockwise a quarter-turn (about ninety degrees) with respect to the introducer sheath 104. In some examples, the quarter-turn revolution may allow the male locking lugs of the introducer lock apparatus 102 to fully engage with the female channels of the introducer sheath 104.

FIG. 4A shows a cross sectional view of a mechanical thrombectomy apparatus 200 as described above in FIGS. 1 and 2A-2C, that may include an outer catheter 210 and an inner shaft 220 (e.g. puller). The inverting tube (e.g., mesh, woven, etc.) is not shown. As shown, the outer catheter 210 may completely surround the inner shaft 220. The outer catheter 210 may move independently from the inner shaft 220. However, fitment between the outer catheter 210 and the inner shaft 220 may be such that there may exist a non-insubstantial friction between the two. In some examples, the mechanical thrombectomy apparatus 200 may include any feasible number of elements surrounded by the outer catheter 210. For example, the outer catheter 210 may surround two or more shafts, wires, tendons, or the like.

In some cases, one or more mechanical devices may be attached to and be operated by a distal end of the mechanical thrombectomy apparatus 200. For example, differential movement between the outer catheter 210 and the inner shaft 220 may operate a distally coupled mechanical device. Operation of the mechanical device may require that differential forces be applied between the outer catheter 210 and the inner shaft 220.

Thus, moving the inner shaft 220 with respect to the outer catheter 210 may require holding or gripping the outer catheter 210 while applying force to the inner shaft 220. In some cases, gripping the outer catheter 210 may be necessary to overcome friction or operate a mechanical device with the inner shaft 220. If the mechanical thrombectomy apparatus 200 is inserted into the introducer sheath 104 and the sheath 106 of FIGS. 3A and 3B, the introducer lock apparatus 102 may be used to grip, hold, and/or lock the outer catheter 210, particularly when articulating or moving the inner shaft 220. In some examples, the introducer lock apparatus 102 may preferentially grip the outer catheter 210 in one direction with respect to the introducer sheath 104.

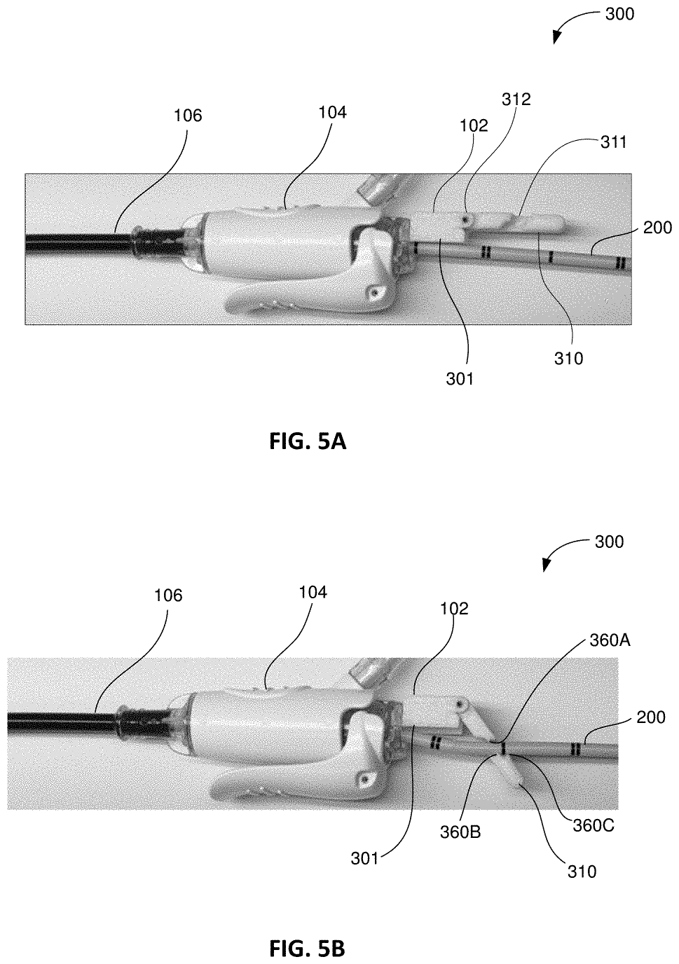

FIG. 4B shows a side view of a catheter 250, in accordance with some examples. The catheter 250 includes an outer catheter 260 and stops 270. The outer catheter 260 may be an example of the outer catheter 210 of FIG. 4A. Furthermore, the outer catheter 260 may enclose or surround any number of shafts, wires, tendons, or the like. The catheter 250 may include stops 270 that may be molded as part of the outer catheter 260. In some other examples, the stops 270 may be coupled or attached to the outer catheter 260. The stops 270 may be slightly larger (in diameter, for example) than the outer catheter 260. Furthermore, the number and spacing of the stops 270 may vary based on implementation. As will be discussed with respect to FIG. 5B, the stops 270 may enable the introducer lock apparatus 102 to better grip the outer catheter 260.

FIG. 5A shows a first view of a catheter controlling system 300, in accordance with some examples. The catheter controlling system 300 may include the introducer lock apparatus 102, the introducer sheath 104, and the sheath 106 of FIGS. 3A and 3B as well as the mechanical thrombectomy apparatus 100 of FIG. 2. As shown, the introducer lock apparatus 102 may be attached to the introducer sheath 104 and may be turned a quarter-turn so that the introducer lock apparatus 102 is attached and/or coupled to the introducer sheath 104. The mechanical thrombectomy apparatus 200 may be inserted through the introducer sheath 104, the introducer lock apparatus 102, and the sheath 106.

The introducer lock apparatus 102 may include a first body 301 and a second body 310. The first body 301 may include male lugs (not shown) to engage with, attach, and/or couple to the introducer sheath 104. As shown, the first body 301 may be colinear and/or coplanar with respect to the mechanical thrombectomy apparatus 200. The second body 310 may include a clamping channel 311 to receive the mechanical thrombectomy apparatus 200.

The first body 301 may be hingeably attached to the second body 310. Thus, a hinge joint 312 that includes hinge pin may couple (e.g., hingeably couple) the first body 301 to the second body 310. In some examples, the introducer lock apparatus 102 may include a spring (not shown), such a spiral spring near or around the hinge pin to bias the second body 310 with respect to the first body 301. For example, the spring may bias a position of the second body 310 in the position shown and also allow the second body 310 to pivot to receive the mechanical thrombectomy apparatus 200 in the clamping channel 311. In some cases, the hinge joint 312 may allow the second body 310 to pivot in a plane perpendicular to an axis or centerline of the mechanical thrombectomy apparatus 200.

FIG. 5B shows a second view of the catheter controlling system 300. In this view, the second body 310 has been pivoted toward the mechanical thrombectomy apparatus 200 such that the mechanical thrombectomy apparatus 200 has been received (positioned) within the clamping channel 311. In some examples, the clamping channel 311 may include one or more teeth 360A-360C that may engage with the outer catheter 210 of the mechanical thrombectomy apparatus 200.

In some examples, the second body 310 may pivot approximately 135 degrees relative the first body, where the second body is fully extended when the second body is in the fully extended position (180 degrees from the first body). Alternatively, if the angle is measure as the change in angle from the fully extended position, the first body is forty-five degrees with respect to the first body 301. This first angle (135 degrees) may be the angle at which the clamping channel has an apparent diameter through the clamping channel in the first axis that is at a maximum when the second body is bent at a first angle relative to the first body to receive the mechanical thrombectomy apparatus 200. Thus, the clamping channel 311 may be configured to accept a diameter of the outer catheter 210 when pivoted at about forty-five degrees with respect to the first body 301.

As the inner shaft 220 is moved proximally with respect to the introducer sheath 104, the outer catheter 210 may also be pulled proximally. Motion of the outer catheter 210 may be due to friction, forces required to operate a mechanical device coupled to a distal end of the mechanical thrombectomy apparatus 200, or the like. (For reference, the proximal direction is to the right and the distal direction is to the left in FIGS. 5A and 5B.) Notably, as the outer catheter 210 tries to move in the proximal direction, the outer catheter 210 increases engagement with the clamping channel 311 (and in some cases the teeth 360A-360C) in the second body 310. However, since the second body 310 is coupled to the first body 301, and since the first body 301 is coupled to the introducer sheath 104, the outer catheter 210 is prevented from moving with respect to the introducer sheath 104 in response to proximal motion of the inner shaft 220.

In some examples, one or more stops (not shown) may be molded or attached to the outer catheter 210. The stops may be larger than the outer catheter 210 and may increase engagement with the clamping channel 311 and/or the teeth 360A-360C compared to just the outer catheter 210.

During use, if the mechanical thrombectomy apparatus 200 is moved in a distal direction with respect to the introducer sheath 104, the second body 310 may also move distally relaxing the grip on the mechanical thrombectomy apparatus 200. Thus, the mechanical thrombectomy apparatus 200 may be preferentially allowed to move distally, while preferentially restricted from moving proximally.

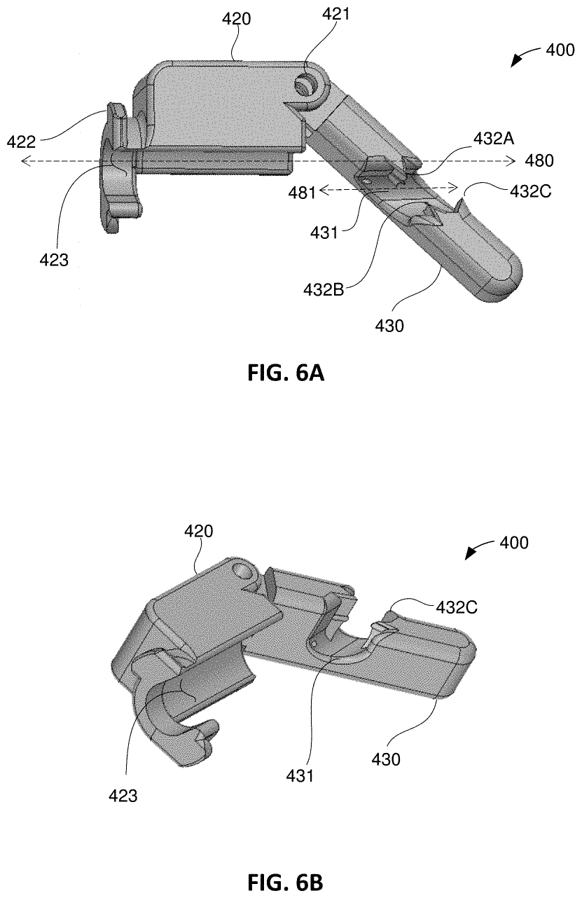

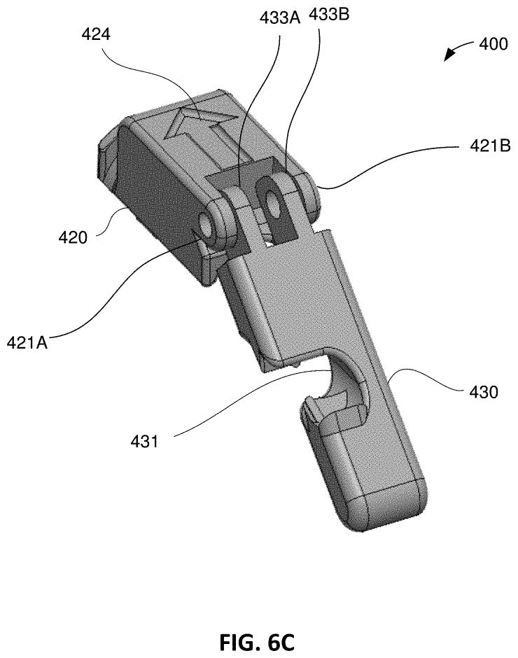

FIG. 6A shows a first view of an introducer lock apparatus 400, in accordance with some examples. The introducer lock apparatus 400 may include a first body 420 and a second body 430. The first body 420 may be an example of the first body 301 and the second body 430 may be an example of the second body 310 of FIGS. 5A and 5B.

In FIG. 6A, the first body includes the catheter channel 423 that is open on one side. The catheter channel includes a long axis 480 extending along the length of the catheter channel. The second body 430 includes a clamping channel 431 formed at an angle through the second body. In FIG. 6A, with the second body bent at an approximate 135 degree angle relative to the first body by bending at the hinge/hinge joint 421. In this configuration the clamping channel has an apparent diameter through the clamping channel in the first axis 481 (that is parallel to the long axis of the catheter seat 480). The apparent diameter is the diameter seen by the outer catheter as it is passed (in line with the catheter channel) through the clamping channel; in FIG. 6A the clamping channel is at a maximum apparent diameter when the second body is bent at a first angle (e.g., approximately 120, 135, 140 etc.) relative to the first body.

The first body 420 may include a hinge joint 421, locking lugs 422, and a catheter channel 423. The hinge joint 421 may enable the first body 420 to hingeably couple and pivot with respect to the second body 430. For example, the hinge joint 421 may enable the first body 420 to pivot about a hinge pin (not shown) on an axis perpendicular to an axis of a catheter, such as the mechanical thrombectomy apparatus 200 (also not shown). The catheter channel 423 may enable the catheter to pass proximate to the first body 420 without binding or any other impediment.

The locking lugs 422 may be male lugs that are configured to fit and mate with a female channel within the introducer sheath 104. Notably, the locking lugs 422 may include a gap or opening that advantageously allows attachment of the introducer lock apparatus 400 to the introducer sheath 104 after a catheter has been inserted in the introducer sheath 104 and/or sheath 106.

The second body 430 may also include a hinge joint (hidden in this view), a clamping channel 431, and teeth 432A-432C. The second body 430 may be coupled to the hinge joint 421 with a hinge pin thereby enabling the second body 430 to pivot about the hinge pin with respect to the first body 420. For example, the hinge joint may enable the second body 430 to pivot about a hinge pin on an axis perpendicular to an axis of the mechanical thrombectomy apparatus 200.

The clamping channel 431 may generally be shaped as a three-sided slot within the second body 430. Two of the sides of the clamping channel 431 may be approximately parallel with each other and disposed at approximately a forty-five degree angle with respect to a surface of the second body 430. A plurality of teeth 432A-432C may be disposed within the clamping channel 431. Although three teeth 432A-432C are shown, in other examples the clamping channel 431 may include any feasible number of teeth. In some examples, at least one of the teeth 432A-432C may be perpendicular to the parallel surfaces of the clamping channel 431. For example, tooth 432C may be relatively perpendicular with respect to the parallel surfaces of the clamping channel 431. The teeth 432A-432C may enable the second body 430 to grip the outer catheter 210 of the mechanical thrombectomy apparatus 200. In some examples, the teeth 432A-432C may grip one or more stops molded or attached to the outer catheter 210. The tooth that projects out of the clamping channel may be referred to as a locking tooth 432C.

FIG. 6B shows a second view of the introducer lock apparatus 400, in accordance with some examples. The second view includes the first body 420 and the second body 430 of the introducer lock apparatus 400. The second view depicts the catheter channel 423 in the first body 420. In some examples, the catheter channel 423 may run the length of the first body 420. The second view also shows the clamping channel 431 on the second body 430.

FIG. 6C shows a third view of the introducer lock apparatus 400, in accordance with some examples. The third view includes the first body 420 and the second body 430 of the introducer lock apparatus 400. The third view depicts hinge joints 433A and 433B of the first body 420. In some examples, the first body 420 may include any feasible number of hinge joints. The first body 420 may also include an arrow 424 to help guide the user during attachment of the introducer lock apparatus 400 to an introducer sheath.

The second body 430 may include hinge joints 433A and 433B. In some examples, the second body 430 may include any feasible number of hinge joints. Another view of the clamping channel 431 is also shown.

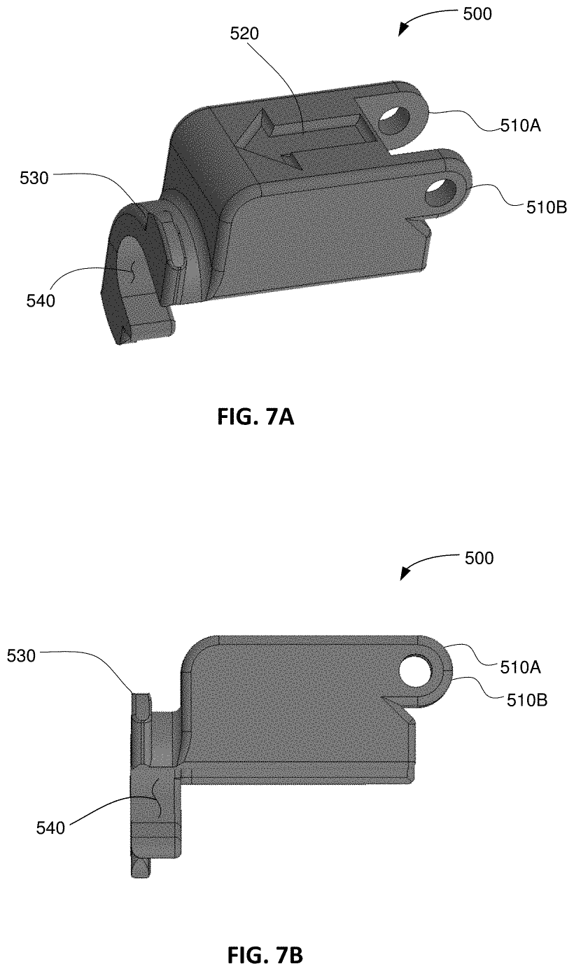

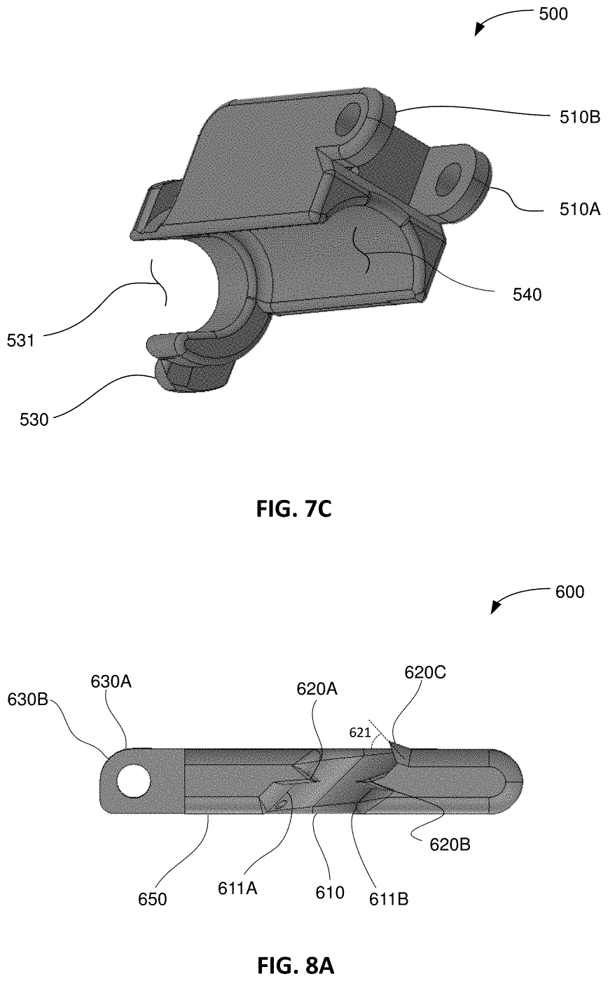

FIG. 7A shows a first view of a first body 500 of the introducer lock apparatus 400 of FIGS. 6A-6C. The first body 500 may include hinge joints 510A and 510B, an arrow 520, locking lugs 530, and a catheter channel 540. The hinge joints 510A and 510B may be examples of the hinge joints 421A and 421B of FIGS. 6A-6C. In some examples, the hinge joints 510A and 510B may receive a hinge pin.

The arrow 520, which may be an example of the arrow 424 of FIG. 6C, may assist the user during insertion of the first body 500 into an introducer sheath. The locking lugs 530 may be examples of the locking lugs 422 of FIG. 6A. Similarly, the catheter channel 540 may be an example of the catheter channel 423 of FIGS. 6A and 6B.

FIG. 7B shows a second view of the first body 500 of the introducer lock apparatus 400 of FIGS. 6A-6C. The second view depicts additional views of the hinge joints 510A and 510B, the locking lugs 530, and the catheter channel 540. FIG. 7C shows a third view of the first body 500 of the introducer lock apparatus 400 of FIGS. 6A-6C. The third view depicts additional views of the hinge joints 510A and 510B, the locking lugs 530, and the catheter channel 540. As shown, the locking lugs 530 may include a gap 531 that advantageously allows attachment of the introducer lock apparatus 400 to an introducer sheath after a catheter has been inserted in the introducer sheath 104 and/or sheath 106.

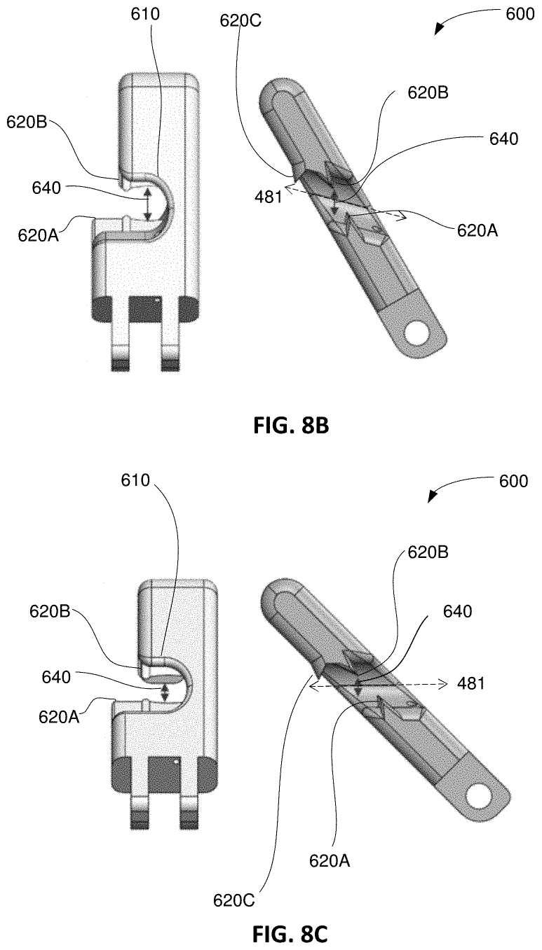

FIG. 8A shows a first view of a second body 600 of the catheter retainer lock 400 of FIG. 6A-6C. The second body 600 may include a clamping channel 610, teeth 620A-620C, and hinge joints 630A and 630B. In some examples, the hinge joints 630A and 630B may receive a hinge pin. The clamping channel 610 may be an example of the clamping channel 311 of FIGS. 5A and 5B and/or the clamping channel 431 of FIGS. 6A-6C. The teeth 620A-620C may be an example of the teeth 432A-432C of FIGS. 6A-6C.

The clamping channel 610 may include approximately parallel surfaces 613A and 613B. The teeth 620A-620C may be disposed on the parallel surfaces 611A and 611B. In particular, one or more of the teeth (620C) may be normal (e.g., perpendicular or ninety degrees) with respect to the parallel surfaces 611A and 611B. Furthermore, the parallel surfaces 611A and 611B may be disposed at approximately at an angle (e.g., between 30-50 degrees, between 35-55 degrees, about forty-five degrees, etc.) with respect to surface 650.

FIGS. 8B and 8C illustrate the apparent diameter through the clamping channel in two configurations. FIG. 8B shows a second view of the second body 600 of the catheter retainer lock 400 of FIGS. 6A-6C. The second view may illustrate the second body 600 positioned at a first angle with respect to the first body 500 (not shown). The teeth 620A-620C may form an opening 640. The apparent distance of the opening 640 may be based at least in part on the angle between the first body 500 and the second body 600. For example, as the second body 600 approaches a ninety degree angle with respect to the first body 500, the opening 640 may become wider with respect to a catheter passing through the clamping channel 610. Conversely, as the second body 600 approaches a zero degree angle with respect to the first body 500, the opening 640 may become narrower, with respect to the catheter passing through the clamping channel 610.

FIG. 8C shows a third view of the second body 600 of the catheter retainer lock 400 of FIG. 4. The opening 640 between teeth 620A-620C is shown. The third view shows the second body 600 at a lesser or decreased angle with respect to the first body 500 than the second body 600 of the second view. Thus, the third view shows the opening 640 being narrower than the opening 640 of the second view. In some examples, as the catheter engages with the teeth 620A-620C, the relative angle decreases between the first body 500 and the second body 600 causing the second body 600 to increase engagement with the catheter. Thus, proximal motion of a catheter in the clamping channel 610 may cause the second body 600 to pivot and have a decreased angle with respect to the first body 500. The decreased angle will decrease the opening 640 in the clamping channel 610 and cause the second body 600 to increase engagement with the catheter.

In this example, the locking tooth 620C is the tooth that extends out of the clamping channel and projects from an outer surface of the second body at a locking angle. As shown in FIG. 8A, the locking angle 621 may be about 45 degrees. In some examples the locking angle is between 35 and 55 degrees (e.g., between 40 and 50 degrees, etc.). The locking tooth may extend in the direction away from which the second body bends, e.g., away from the angle between the first body and the second body when the second body is engaged with the outer catheter, similar to that shown in FIGS. 6A and 6B. The locking tooth 620C is present on the upper surface of the second body (second arm).

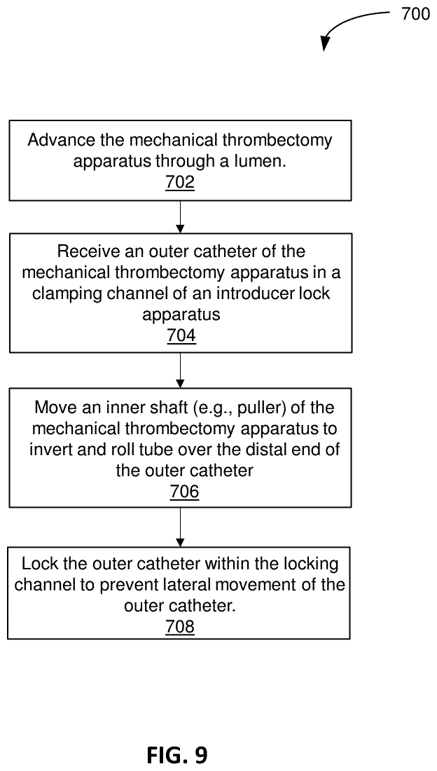

FIG. 9 is a flowchart depicting an example of one method 700 for controlling a mechanical atherectomy apparatus (system). Some examples may perform the operations described herein with additional operations, fewer operations, operations in a different order, operations in parallel, and some operations differently. The method 700 may be used to control movement of a mechanical atherectomy apparatus, particularly when motion is applied to one or more internal shafts of the outer catheter of the mechanical atherectomy apparatus.

In FIG. 9, the method 700 may begin as a mechanical atherectomy apparatus is advanced through a lumen in block 702. For example, a clinician may insert and guide an outer catheter of a mechanical atherectomy apparatus into a body lumen of a patient and adjacent to a clot.

Next, in block 704 an outer catheter is received in a clamping channel of an introducer lock apparatus. For example, an introducer lock apparatus may include a first body removably attached to an introducer sheath and a second body hingeably coupled to the first body. The second body may include a clamping channel that can receive the catheter, and in particular receive an outer catheter. In some examples, the clamping channel may be a slot disposed at approximately a forty-five degree angle with respect to an outer surface of the second body.

Next in block 706, the clinician may move an inner shaft of the mechanical atherectomy apparatus within the mechanical atherectomy apparatus in order to pull the inverting tube of the mechanical atherectomy apparatus into the outer catheter, rolling over the distal end opening of the outer catheter, but compressing the outer catheter, and potentially driving it proximally. For example, the clinician may move the inner shaft proximally with respect to the introducer sheath. As a result, the catheter in general, and the outer catheter in particular, may also move in a proximal direction.

Next in block 708, the clamping channel engages with the outer catheter in response to motion of the inner shaft. Since the second body (which includes the clamping channel) is coupled to the first body, and since the first body is coupled to the introducer sheath, motion of the catheter with respect to the introducer sheath may be reduced or limited as the clamping channel engages. For example, the clamping channel may increase a grip of the catheter as the inner shaft of the catheter moves in a proximal direction. In some examples, the clamping channel may include one or more teeth to engage and or grip the outer catheter. In other examples, the catheter may include one or more stops on the outer catheter to engage with the clamping channel.

When a feature or element is herein referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected”, “attached” or “coupled” to another feature or element, it can be directly connected, attached, or coupled to the other feature or element or intervening features or elements may be present. In contrast, when a feature or element is referred to as being “directly connected”, “directly attached” or “directly coupled” to another feature or element, there are no intervening features or elements present. Although described or shown with respect to one embodiment, the features and elements so described or shown can apply to other embodiments. It will also be appreciated by those of skill in the art that references to a structure or feature that is disposed “adjacent” another feature may have portions that overlap or underlie the adjacent feature.

Terminology used herein is for the purpose of describing particular embodiments only and is not intended to be limiting of the invention. For example, as used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items and may be abbreviated as “/”.

Spatially relative terms, such as “under”, “below”, “lower”, “over”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under” or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly. Similarly, the terms “upwardly”, “downwardly”, “vertical”, “horizontal” and the like are used herein for the purpose of explanation only unless specifically indicated otherwise.

Although the terms “first” and “second” may be used herein to describe various features/elements (including steps), these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings of the present invention.

Throughout this specification and the claims which follow, unless the context requires otherwise, the word “comprise”, and variations such as “comprises” and “comprising” means various components can be co jointly employed in the methods and articles (e.g., compositions and apparatuses including device and methods). For example, the term “comprising” will be understood to imply the inclusion of any stated elements or steps but not the exclusion of any other elements or steps.

In general, any of the apparatuses and methods described herein should be understood to be inclusive, but all or a sub-set of the components and/or steps may alternatively be exclusive and may be expressed as “consisting of” or alternatively “consisting essentially of” the various components, steps, sub-components, or sub-steps.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical values given herein should also be understood to include about or approximately that value unless the context indicates otherwise. For example, if the value “10” is disclosed, then “about 10” is also disclosed. Any numerical range recited herein is intended to include all sub-ranges subsumed therein. It is also understood that when a value is disclosed that “less than or equal to” the value, “greater than or equal to the value” and possible ranges between values are also disclosed, as appropriately understood by the skilled artisan. For example, if the value “X” is disclosed the “less than or equal to X” as well as “greater than or equal to X” (e.g., where X is a numerical value) is also disclosed. It is also understood that the throughout the application, data is provided in a number of different formats, and that this data, represents endpoints and starting points, and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

Although various illustrative embodiments are described above, any of a number of changes may be made to various embodiments without departing from the scope of the invention as described by the claims. For example, the order in which various described method steps are performed may often be changed in alternative embodiments, and in other alternative embodiments one or more method steps may be skipped altogether. Optional features of various device and system embodiments may be included in some embodiments and not in others. Therefore, the foregoing description is provided primarily for exemplary purposes and should not be interpreted to limit the scope of the invention as it is set forth in the claims.

The examples and illustrations included herein show, by way of illustration and not of limitation, specific embodiments in which the subject matter may be practiced. As mentioned, other embodiments may be utilized and derived there from, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is, in fact, disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

What is claimed is:

1. An introducer lock apparatus, the apparatus comprising:
a first body comprising a catheter channel extending proximally to distally in a first axis, wherein the catheter channel is configured to seat a catheter therein;
a sheath connector at a distal end of the first body, the sheath connector at least partially surrounding the catheter channel and is configured to releasably engage with an introducer sheath;
a second body hingeably coupled to the first body; and
a clamping channel extending through a lateral side of the second body, the clamping channel forming an angle relative to the second body, so that the clamping channel has an apparent diameter through the clamping channel in the first axis that is at a maximum when the second body is bent at a first angle relative to the first body, further wherein the apparent diameter through the clamping channel in the first axis decreases as the angle relative to the second body increases.

2. The apparatus of claim 1, further comprising one or more teeth extending into the clamping channel from a wall of the clamping channel.

3. The apparatus of claim 1, further comprising a locking tooth extending out of the clamping channel and projecting from an outer surface of the second body at a locking angle.

4. The apparatus of claim 1, wherein a central axis extending along the clamping channel is parallel with the first axis when the apparent diameter is at the maximum.

5. The apparatus of claim 1, wherein the catheter channel is open along an outward-facing side of the first body.

6. The apparatus of claim 5, wherein the clamping channel forms a C-shaped channel having a side opening that does not align with the opening of the catheter channel.

7. The apparatus of claim 5, wherein the sheath connector only partially surrounding the catheter channel.

8. The apparatus of claim 1, wherein the sheath connector comprises a Leur fitting.

9. The apparatus of claim 1, wherein the sheath connector comprises a partial Leur fitting.

10. The apparatus of claim 1, further comprising a stop on the first body, limiting the angle between the first body and the second body.

11. The apparatus of claim 1, wherein the first angle relative to the first body is between 120-130 degrees.

12. The apparatus of claim 1, wherein the second body comprises a rigid arm.

13. The apparatus of claim 1, further comprising a bias configured to hold the second body at the first angle relative to the first body.

14. A method of operating a mechanical atherectomy system, the method comprising:
advancing a distal end of an outer catheter of the mechanical atherectomy system through a sheath and into a body lumen;
securing the outer catheter within an introducer lock apparatus coupled to the proximal end of the sheath so that a first region of the outer catheter is seated in a catheter channel of a first body extending in a first axis, and a second region of the outer catheter proximal to the first region is held within a clamping channel of the introducer lock apparatus, wherein the clamping channel is formed through a lateral side of a second body that is hingeably coupled to the first body;
pulling an inner shaft of the mechanical atherectomy system proximally to invert a tubular member over a distal end of the outer catheter so that it rolls over the distal end of the outer catheter and into the outer catheter; and
locking the outer shaft to the sheath by changing the angle of the second body when the inner shaft is driven proximally by pulling the inner shaft, so that an apparent diameter through the clamping channel in the first axis decreases.

15. The method of claim 14, wherein pulling the inner shaft proximally applies a proximal force on the outer catheter that changes the angle of the second body to lock the outer shaft to the sheath.

16. The method of claim 14, wherein locking the outer shaft to the sheath comprise gripping an outer surface of the outer catheter with one or more teeth extending from a wall of the clamping channel and into the clamping channel.

17. The method of claim 14, wherein locking the outer shaft to the sheath comprises gripping an outer surface of the outer catheter with one or more locking teeth extending from an outer surface of the second body at a locking angle.

18. The method of claim 14, wherein the method is performed by a user holding the sheath with a first hand and a proximal end of the inner member with a second hand.

19. The method of claim 17, wherein the user does not hold the outer catheter.

20. The method of claim 14, wherein locking the outer shaft to the sheath comprises gripping one or more stops on an outer surface of the outer catheter.
